(19) United States
(12) Patent Application Publication
(10) Pub. No.: US 2022/0193384 A1
(43) Pub. Date: Jun. 23, 2022

(54) CUTANEOUS DEVICE FOR STORING AND RELEASING MOLECULES, AND CORRESPONDING METHOD

(71) Applicants: UNIVERSITE DE LILLE, Lille (FR); ECOLE CENTRALE DE LILLE, Villeneuve d'Ascq (FR); CENTRE NATIONAL DE LA RECHERCHE SCIENTIFIQUE, Paris (FR); YNCREA HAUTS DE FRANCE, Lille (FR); UNIVERSITE POLYTECHNIQUE DES HAUTS DE FRANCE, Valenciennes (FR); UNIVERSITE CATHOLIQUE DE LOUVAIN, Louvain-LaNeuve (BE)

(72) Inventors: Sabine SZUNERITS, Villeneuve d'Ascq (FR); Rabah BOUKHERROUB, Villeneuve d'Ascq (FR); Sorin MELINTE, Bruxelles (BE)

(21) Appl. No.: 17/601,917

(22) PCT Filed: Mar. 24, 2020
(86) PCT No.: PCT/EP2020/058239

§ 371 Date: Oct. 6, 2021

(30) Foreign Application Priority Data
Apr. 8, 2019 (EP) 19167905.9

(51) Int. Cl. A61M 37/00; A61K 9/70; A61K 38/28

(52) U.S. Cl. CPC A61M 37/00; A61K 9/703; A61M 2205/3653; A61M 2037/0007; A61K 38/28

(57) ABSTRACT

The present invention relates to a cutaneous device (2) for the storage and release of molecules, in particular therapeutic molecules, comprising: —a support (6), —a matrix (8) configured to store and release the molecules as a function of the temperature, and —an electro-resistive layer, disposed between the support and the matrix, intended to be subjected to a voltage and comprising at least one thin microporous and/or nanoporous metal layer. The present invention also relates to a transcutaneous delivery device and the corresponding methods of manufacture and use.

### BACKGROUND OF THE INVENTION

The present invention relates to transcutaneous release devices for molecules, in particular therapeutic molecules. More particularly, the invention relates to a cutaneous device for storing and releasing molecules, in particular therapeutic, controlled by a voltage.

Controlled release of therapeutic products over long periods of time is currently considered to be one of the most promising biomedical technologies for the treatment of diseases requiring medications that must be administered intermittently on demand and over a longer period of time. For this purpose, a huge body of research is dedicated to the development of automatic drug delivery systems. Nevertheless, this approach is complex because it requires the choice of a biomaterial capable of undergoing specific chemical and/or physical modifications upon experiencing an external stimulus or in vivo, as well as an appropriate route of administration.

Although drug molecules can be inserted into polymeric or other nanostructures to protect them, at least partially, from degradation in the stomach, oral administration does not seem appropriate for controlled release and on long-term.

Compared with oral delivery systems, transdermal approaches have the advantage of not having to cross the intestine and the liver. In addition, transdermal administration is generally better accepted by patients, and reduces side effects. It also avoids the pain and safety problems associated with injecting drugs using a syringe. Transdermal administration using cutaneous patches is also suitable for a controlled and continuous release of therapeutic agents over a prolonged period, which results in an improvement of the therapeutic efficacy. The main areas of use for transdermal drug delivery systems include: chronic pain, cardiovascular disease, hormone replacement therapy, local skin analgesia, central nervous system (CNS) disease, cancer, and more like madness.

While administering drugs via the skin is an attractive option, normal skin is, nevertheless, a significant barrier to most standard drugs, other than those that are lipophilic (fat soluble) and low molecular weight. Such remarkable barrier properties are due in large part to the stratum corneum, the outermost layer of the epidermis, which has a thickness of 10 to 40 μm. The stratum corneum consists of dead cells (corneocytes) embedded in a lipid-enriched extracellular matrix organized into a lamellar structure with elongated and tortuous pathways that increase the diffusion length of drugs through it. The lipid matrix is composed of ceramides, cholesterol and fatty acids, with ceramides being necessary for the lamellar organization of the stratum corneum, cholesterol promoting the mixing of different lipid species and free fatty acids responsible for hydrophobic/lipophilic properties. The resulting low permeability of the stratum corneum to water-soluble drugs is, therefore, an obstacle to transdermal administration by passive diffusion of drugs.

From a physicochemical point of view, for a medicament to be administered transdermally, it is necessary that it has certain characteristics such as high lipophilicity, low molecular weight (for example less than 500 Dalton), sufficient solubility in water at a pH between 6 and 7.4, and a suitable pKa. Thus, significant efforts have been made to develop new approaches to improve transdermal drug delivery.

Such approaches are intended to provide an alternative to conventional passive systems and broaden the spectrum of transdermally administrable drugs, including non-lipophilic and hydrophilic drugs, or larger molecules such as peptides, protein antibodies or nucleic acids. In addition, controlled release on demand is also desired.

An example of an approach is iontophoresis in which a physiological acceptable electric current (0.1-1 A·cm⁻²) is applied for minutes to hours to improve the rate of drug delivery through the skin. Thus, an iontophoretic patch for lidocaine/epinephrine delivery, is used in combination with very short but high (100 V) voltage pulses. The voltage pulses are used to induce rearrangements of the lipid structures of the stratum corneum, which leads to the formation of pores and facilitates the administration of the drug.

However, such an approach is not suitable for delivering larger molecules. Moreover, the amount of drug delivery is also limited by the maximum current that may be applied until the pain level is reached.

An example of electrochemical method is described in the document Electrochemically triggered release of human insulin from an insulin-impregnated reduced graphene oxide modified electrode (TEODORESCU, July 2015). The document relates to a patch for delivering, in a controlled manner, insulin in aqueous media. The patch comprises a gold electrode on which is deposited a matrix of reduced graphene oxide loaded insulin. The application of a current by the gold electrode allows a local modification of the pH and the release of the insulin stored in the matrix. This document demonstrates the feasibility of storing and releasing insulin in a patch but does not give any indication of the feasibility of transdermal delivery of insulin.

However, it has not yet been shown that such an electrochemical method would be useful for transdermal drug delivery.

Another way to more easily cross the stratum corneum is through application of heat. The heat induces a destabilization of the stratum corneum that allows the drug to cross. Most applications using photothermal agents or photothermal agents based on graphene are related to the release of anticancer drugs (such as doxorubicin or paxlitacel).

The documents Photothermally triggered on-demand insulin release from rGO modified hydrogels (TEODORESCU, October 2016) and Transdermal skin patch based on rGO: a new approach for photothermal triggered permeation of ondansetron across porcine skin (TEODORESCU, November 2016) concern the controlled release respectively of insulin and ondansetron, stored in a matrix of reduced graphene oxide (rGO), by photothermal control. Specifically, hydrogel/rGO and Kapton/rGO-based photothermal heating patches allow the release and transdermal delivery of insulin and ondansetron through the application of near infrared light, for example a laser at 980 nm. The light makes it possible to locally increase the temperature of the matrix, and thus to release the insulin and ondansetron molecules respectively, in a controlled manner without affecting the insulin and ondansetron activities (TEODORESCU, November 2016).

Document Flexible nanoholey patches for antibiotic-free treatments of skin infections relates to a skin patch allowing an efficient treatment of subcutaneous wound infections via photothermal irradiation.

However, the main disadvantage of photothermal control is the use of a near-infrared laser which is not portable and which may be dangerous, so that the portability of such a method is limited.

### OBJECT AND SUMMARY OF THE INVENTION

The present invention aims to solve the various technical problems mentioned above. In particular, the present invention aims to provide an easier and a safer method, and the corresponding device, for storing and releasing molecules, including therapeutic molecules, in a controlled manner through an electro-thermal control. In particular, the present invention aims at providing a method, and the corresponding portable device, in which the electro-thermal control voltage of the storage or the release of the drug molecules is a low voltage.

Thus, according to one aspect, a cutaneous device is provided for storing and releasing molecules, in particular therapeutic molecules, comprising:
a support,
a matrix configured to store and release the molecules as a function of the temperature, and
an electroresistive layer, placed between the support and the matrix, intended to be subjected to a voltage.

Thus, thanks to the device according to the present invention, it is possible to administer molecules transdermally, in particular therapeutic molecules, without the use of light but only with heat. Indeed, in the prior art devices, it was considered that the infra-red light has to be used both to heat the cutaneous device but also to heat the first layers of the user skin: the infra-red light is a wave that may penetrate the skin over a small thickness, allowing a warming of the skin over this thickness, which facilitates the flow of the molecules through such a thickness of the skin.

It has been discovered that an electro-thermic cutaneous device may also be used to administer molecules transdermally, and that the heating of the skin by the cutaneous device is enough to achieve a flow of the molecules through the skin.

Preferably, the electro-resistive layer comprises at least one microporous and/or nanoporous thin metallic layer.

Thanks to the electro-resistive layer, or electro-thermal skin device storage and release of molecules, it is possible to heat the matrix storing the drug molecules and to release them. The release of the drug molecules is carried out in a controlled manner, by the application, or not, of a voltage at the ends of the electro-resistive layer in order to create a temperature increase by Joule effect. The increase in temperature makes it possible on one hand to release the molecules stored in the matrix but also to improve the passage of said molecules through the skin, in particular for molecules with a high molecular weight. More particularly, the use of a thin metal layer, and in particular of a thin gold layer, in the cutaneous device is made possible through the addition of micro- and/or nano-holes in the thin metal layer. The microporous and/or nanoporous thin metal layer allows to obtain a temperature increase, for a given current passing through the thin layer, together with a better mechanical strength in comparison to a full uniform thin metal layer, which may be too brittle fur being used in a cutaneous device. The invention, therefore, makes it possible to have a cutaneous device for storing and releasing molecules that can be controlled easily, by applying an electrical voltage, and that only requires a low-voltage electrical source to release the molecules. It is thus possible to improve the autonomy of the device which is intended to be worn by the user throughout the day and/or night for a regular release or at fixed intervals of the therapeutic molecules.

The skin device is, for example, a patch to be applied to the skin and intended to deliver, in a controlled manner, therapeutic molecules stored therein when it is connected to a control means capable of supplying a low voltage.

The therapeutic molecules can be drug molecules.

Preferably, the microporous and/or nanoporous thin metallic layer comprises a network of pores, or holes, micrometric and/or nanometric. The micrometric and/or nanometric pores make it possible to reduce the mechanical weakness of the thin layer, with respect to a thin layer devoid of such pores, which is a main feature for a thin layer intended to be used in a cutaneous device.

Preferably, the cutaneous device, and particularly the electroresistive layer, is configured to dissipate, as heat, electrical power densities below 200 mW per centimeter square. Such electrical power densities are chosen for attaining the useful operational temperatures, between the body temperature and the maximum temperature of 52° C. The temperature of the device rises and attains the required steady state value for such corresponding applied electrical power, proportional to the varying electrical biases and the magnitude of the electrical current flowing through the device.

Preferably, the metal thin layer comprises a material chosen from: gold, silver, copper, nickel, platinum, titanium and carbon. The materials considered are thermal conductors having an electrical resistance value making it possible to obtain the desired temperature increase by Joule effect under a low applied voltage.

Preferably, the cutaneous device is configured to reach a temperature of between 30° C. and 70° C., preferably between 40° C. and 60° C., when the electro-resistive layer is subjected to a voltage of between 1V and 10V, preferably between 2V and 5V. Such temperature increases make it possible, on the one hand, to trigger the release of the stored molecules, and on the other hand to promote their diffusion through the skin. In addition, the applied voltages remain low and facilitate the design and use of a transcutaneous delivery device as described below.

Preferably, the micrometric and/or nanometric pores have a diameter between 1 nm and 1 μm, preferably between 5 nm and 500 nm, preferably between 10 nm and 200 nm, and more preferably between 15 nm and 25 nm. The choice of the pore diameter is determined in particular according to the mechanical strength of the material used to form the thin metal layer and depending on the voltage that will be applied to obtain the desired temperature increase.

Preferably, the cutaneous device also comprises the molecules stored and released by the device, for example insulin. In this embodiment, the molecule to be stored and released, for example insulin, is previously loaded onto the cutaneous device for which it is only necessary to apply a voltage to release the molecules.

Preferably, the molecules are therapeutic molecules having a molecular weight of between 500 Da and 10 kDa, preferably between 4000 Da and 8000 Da, preferably between 5000 Da and 7000 Da. The purpose of the cutaneous device is to allow molecules of relatively high molecular weight to be able to pass through the skin of the user. Thus, the application of a voltage, and not only of infra-red light, to obtain an increase in temperature is intended to facilitate the crossing of the skin by molecules for which the molecular weight does not allow them to cross naturally.

Preferably, the therapeutic molecules may be chosen among: hormones, proteins such as insulin or ovalbumin, peptides, painkillers, anticancer drugs, antibiotics . . . .

Preferably, the matrix comprises at least one component chosen from: reduced graphene oxide, porous reduced graphene oxide, doped reduced graphene oxide (N-doped, . . . ) and/or one or more reduced graphene oxide derivatives, and/or one or more graphene-based composites such as G/MoS₂. The reduced graphene oxide is already known and used to store and release therapeutic molecules, including insulin. Its use therefore makes it possible to have a reliable and proven behavior for the storage and the release of the therapeutic molecules.

Preferably, the support is made of polymer, for example polyimide, polyurethane, polymethylmethacrylate or polyethylene. For example, the support can be in Kapton®. The support is chosen for its mechanical strength, while allowing a certain flexibility compatible with its use for a patch intended to be placed on the skin. In addition, the support is an electrical insulator so as not to interfere with the thin metal layer and the power supply thereof.

According to another aspect, there is also provided a device for the transdermal release of molecules, in particular therapeutic molecules, comprising a cutaneous device as described above, and means of supplying the electro-resistive layer configured to subject the electro-resistive layer to a voltage of between 1V and 10V, preferably between 2V and 5V. The transcutaneous delivery device thus comprises, on the one hand, the cutaneous device controllable with a low voltage, and on the other hand the power supply of the cutaneous device to be able to control the release, or not, of the molecules stored in the cutaneous device.

The power supply may be a DC power supply supplying a voltage of up to 10V.

The electro-resistive layer is thus connected directly to the terminals of the supply means, and is therefore directly subjected to the voltage imposed by the supply means. In particular, the thin metallic layer is not an electrode connected to a single terminal of a supply means for presenting a given potential value, but rather a resistive element comprising two terminals, for example two ends, connected to a potential difference imposed by the supply means, so that a current flows in the thin metal layer, between said two terminals.

Preferably, the means for supplying the electro-resistive layer are configured to apply a voltage and a current to the electroresistive layer so as to allow the dissipation as heat, by the electroresistive layer, of electrical power densities below 200 mW per centimeter square.

In another aspect, there is also provided a method of manufacturing a skin device as described above, wherein the thin metal layer is formed by colloidal lithography, for example using polymeric particles. Colloidal lithography is intended to allow the formation of the microporous and/or nanoporous thin layer. According to such a method, a monolayer of microbeads and/or nanobeads, are deposited on a substrate and then reduced in size.

Preferably, the polymeric particles are polystyrene particles. Advantageously, the particles have a diameter of between 100 nm and 1000 nm. Preferably, the size of the particles is adjusted (here reduced), for example by plasma etching. Advantageously, in the case where the particles have a diameter of between 100 nm and 1000 nm, the reduction will bring the particles to a diameter between 500 nm and 700 nm, and for example, preferably to a diameter equal to 630 nm. A thin metallic layer of thickness less than the diameter of the etched beads, for example 50 nm, is then deposited on the substrate with said beads.

Finally, the beads are dissolved, for example in chloroform, to obtain a thin metal layer having micrometric and/or nanometric holes in the thin metal layer.

Preferably, the thin layer is formed on the support. The thin nanoporous and/or microporous metal layer is thus directly formed by depositing on the support.

Preferably, the configured matrix is then deposited to store and release molecules, in particular therapeutic molecules, as a function of temperature, on the thin metal layer, for example by drop-casting, by spin casting, by electrophoretic deposition or by spraying. The matrix thus deposited may be empty, that is to say without molecules stored inside, or on the contrary previously loaded with molecules, including therapeutic.

According to a first embodiment, before the deposition of the matrix on the thin metal layer, the molecules, in particular the therapeutic molecules, are stored in the matrix, for example by implementing the following successive steps:
mixing and stirring, for example by applying sound waves, of the matrix with the molecules, then
separation by precipitation of the non-stored molecules in the matrix, then
evacuation wash of non-stored molecules.

According to this embodiment, the matrix is loaded with the molecules, in particular therapeutic molecules, and is then deposited with the molecules stored on the thin metal layer. Such an implementation can be considered especially in the case where the cutaneous device is intended for a single use: in this case, the matrix may not necessarily be rechargeable in molecules, including therapeutic, and the loading of the matrix could then be performed during the manufacture of the matrix, before its deposition on the thin metal layer.

According to another embodiment, after the deposition of the matrix on the thin metal layer, the molecules, particularly therapeutic molecules, are stored in the matrix, for example by immersing the cutaneous device in a solution containing the molecule of interest.

In this embodiment, it is considered that the matrix can be reloaded with molecules, in particular therapeutic molecules. In this case, the storage of the molecules in the matrix can be carried out indifferently before or after the deposition of the matrix on the thin metal layer. The matrix can therefore be deposited without molecules stored inside, then be loaded once the skin device is made, similar to the subsequent loadings that will be performed each time the matrix is discharged.

The matrix can be refilled for example by immersion it in a solution containing molecules, in particular therapeutic molecules. The immersion of the matrix may be accompanied by a mixing step, possibly with the application of ultrasonic waves. After loading the matrix with the molecules, the matrix can be separated from the solution by precipitation and then washed to evacuate the non-stored molecules in the matrix.

In another aspect, it is finally proposed a method of transcutaneous release of molecules from a cutaneous device as described above, in which the cutaneous device is applied to the skin of an individual, then the electro-resistive layer is subjected to a voltage of between 1V and 10V, preferably between 2V and 5V, so that the temperature of the cutaneous device is between 30° C. and 70° C., preferably between 40° C. and 60° C.

It is also proposed a method of transcutaneous release of molecules from a cutaneous device as described above, in which the cutaneous device is applied to the skin of an individual, then the electro-resistive layer is subjected to a voltage of between 1V and 10V, preferably between 2V and 5V, and to a current so as to get a dissipation, as heat, of electrical power densities below 200 mW per centimeter square.

In this embodiment of the transcutaneous release device, it is sufficient to apply a low voltage to the electro-resistive layer, to obtain an increase in temperature of the cutaneous device and therefore a release of molecules, including therapeutic, stored in the matrix.

Preferably, the voltage is applied to the electro-resistive layer continuously, in response to a command from the user, or at regular intervals, for example for a period of between 1 and 10 minutes, every 1 to 6 hours. Thus, the control of the transcutaneous release device can be done according to a memorized protocol and implemented by an electronic device, or can be done by order of the user who triggers, on request, a release of therapeutic molecules.

### BRIEF DESCRIPTION OF THE DRAWINGS

The invention and its advantages will be better understood on reading the detailed description of a particular embodiment, taken by way of non-limiting example and illustrated by the appended drawings in which:

FIG. 1: Schematic representation of a transcutaneous delivery device according to the invention.

FIG. 2: Diagrammatic representation, in section, of the cutaneous device of the transcutaneous release device illustrated in FIG. 1.

FIG. 3: Flowchart of an example of manufacture of the cutaneous device according to the invention, and

FIG. 4: Flowchart of an exemplary implementation of the transcutaneous delivery device according to the invention.

FIG. 5: (A) Schematics of the electrothermal patch based on reduced graphene oxide-coated gold nanoholes array formed by colloidal lithography on polyimide (Kapton) films. The Kapton substrate (K), the metallic layer (Au NHs), the rGO coating and the insulin are not shown to scale; (B) List of eight molecuels tested.

FIG. 6: (A) Structures of different gold nanoholes arrays deposited on Kapton formed by colloidal litoraphy and changing the etching parameters (B) Thermal image displaying the temperature distribution across the surface of the electrothermal patch; (C) Steady-state temperature of K/Au NHs patches as a function of applied voltage; (D) Temperature vs. applied voltage for a K/Au NHs device flat and bent as a function of applied voltage; (E) Temperature profile over time upon the application of 1V for 75 s for K/Au NHs and K/Au NHs-rGO; (F) Temperature vs. applied voltage for a K/Au NHs-rGO device flat and bent as a function of applied voltage.

FIG. 7: Calibration curves together with HPLC sensograms (A-F) for different drugs investigated in the passive and active form: (A) caffeine, (B) metformin, (C) 5-fluorouracil, (D) ampicillin, (E) cefepime, (F) doxorubicin. In the case of insulin (G) a commercial ELISA insulin kit was used (Invitrogen (Ref #KAQ1251) with analytical sensitivity of 0.17 μIU/mL and an assay range of 0.1-250 μIU/mL).

FIG. 8: (A) Amount of DOX and insulin (stock solution of 100 μg mL⁻¹ were used for loading) permeated under electrothermal activation for 10 min as a function of applied voltage (0.6V=35° C., 0.8V=40° C.; 0.85V=45° C., 0.95V=50° C., 1.00V=52° C., 1.05V=55° C., 1.15V=60° C.)); Hematoxyline eosine staining showing the histological changes of mouse skin after heating for 10 minutes at (B) 52° C., (C) 55° C., (D) 60° C., the thickening of the epidermis and destruction of the sweat gland at 60° C. are observed. The white arrows indicate the thickness of the epidermis.

FIG. 9: In vivo experiments testing the therapeutic effect. (A) Photograph of mice carrying electrothermal patch together with photo of mouse skin after electrothermal treatment (left) together with histological images using Giesma coloration, (B) Change in blood glucose level (BGL) upon intraperitoneal injection (IP) and subcutaneous injection of insulin (0.032 UI, 1.12 μg); (C) Change of relative BGL (%) with time. The control group was not given insulin (6 IU) but 1V was applied for 10 min. The positive control group was treated with an insulin loaded electrothermal patch and activated for 10 min at 1V. The results of SP injection of insulin (0.032 IU) were added, variation of activation time, keeping the same potential: all data points represent the average±standard deviation of measurements in 4-6 different mice.

### DETAILED DESCRIPTION OF THE INVENTION

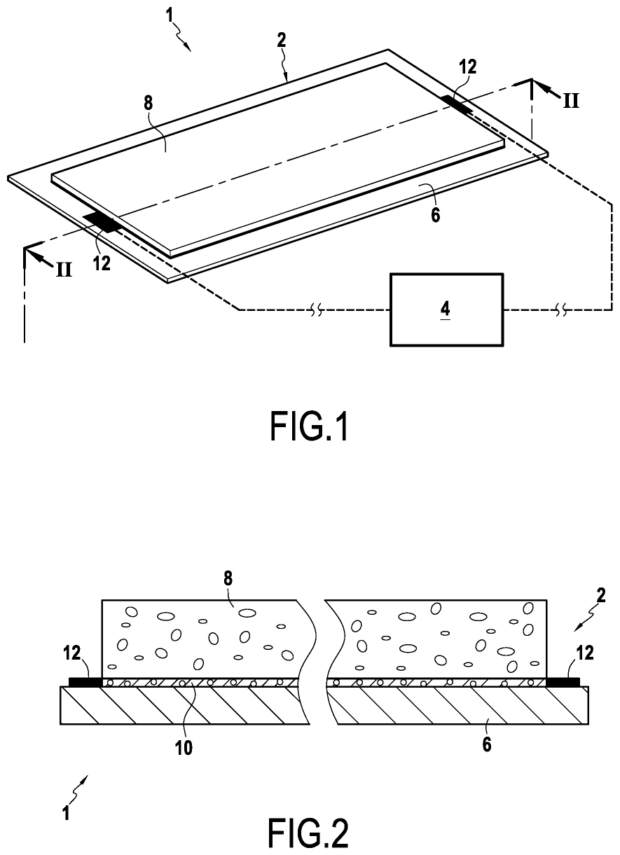

FIG. 1 schematically illustrates a transcutaneous delivery device 1 according to the present invention.

The device 1 comprises a cutaneous device 2 and a power supply means 4 configured to provide a low value voltage, for example between 1V and 10V, and preferably between 2V and 5V.

The cutaneous device 2 comprises in particular a support 6 and a matrix 8 for storing and releasing molecules, in particular therapeutic molecules, for example insulin molecules.

As can be seen in FIG. 2 which represents the cutaneous device 2 of FIG. 1 in section, the cutaneous device 2 also comprises a thin microporous and/or nanoporous metal layer 10 placed between the support 6 and the matrix 8. As will be described below, the thin metal layer 10 is configured to be electro-resistive, and is, for this purpose, electrically connected to the power supply means 4 via in particular contacts 12 located on the cutaneous device 2 and for transmitting the voltage imposed by the power supply means at two opposite points of the thin metal layer 10, for example at the ends of the thin metal layer 10. Thus, the thin metal layer 10 constitutes a resistive element in which the current delivered by the power supply means 4 circulates from one end to another, to create, by Joule effect, an increase in temperature of the surface of the layer 10. The electrosensitive layer of the cutaneous device thus comprises a thin nanoporous and/or microporous metal layer 10, and electrical contacts 12 for connection to a supply means.

More specifically, the support 6 is a thin and flexible support adapted for use as a patch to be applied to the skin, and having a high temperature resistance and a high chemical resistance.

The support 6 may thus be a polymeric film, in particular polyimide, polyurethane, polymethylmethacrylate or polyethylene. By way of example, the support 6 may be a Kapton® film, for example of square shape of 10 mm by 10 mm.

The matrix 8 is a matrix for the storage and release of molecules, in particular therapeutic molecules, which may have a high molecular weight, for example greater than 500 Da or even greater than 4000 Da, such as insulin. The matrix 8 may comprise reduced graphene oxide, or one or more reduced graphene oxide derivatives, which make it possible to store and release molecules such as insulin. In particular, the release of the molecules can be controlled by the temperature of the matrix 8, an increase in temperature causing a release of the stored molecules. The matrix 8 may have a micrometric thickness.

The matrix 8 can be configured to be rechargeable so that it can continue to use the skin device 2 even after it has been discharged, by implementing a step of reloading the matrix 8 with molecules. Alternatively, the matrix 8 can be provided originally loaded, and can be used only once, until the matrix 8 is empty.

The reduced graphene oxide is already known and used to store and release molecules, especially therapeutic molecules, for example anti-cancer molecules. In particular, reduced graphene oxide has the advantage of being able to be recharged with molecules efficiently and simply. In particular, it is possible to obtain an insulin loading capacity of up to 90%. The reduced graphene oxide, or its derivatives, is therefore a particularly interesting material for reusable cutaneous devices.

The thin metal layer 10 is intended to provide heat when subjected to an electrical voltage. More specifically, and in order to be able to provide the wanted heat when subjected to a low electrical voltage, for example less than 10V and preferably less than 5V, the thin metal layer 10 is provided in nanoporous and/or microporous form.

The material of the thin metal layer 10 is an electrical conductor, and can therefore be selected from the following materials: gold, silver, copper, nickel, platinum, titanium, carbon, etc. Preferably, the thin metal layer 10 is a nanoporous layer of gold.

The pores, or holes, of the thin metal layer 10 preferably have a diameter of between 1 nm and 1000 nm, preferably between 5 nm and 500 nm, preferably between 10 nm and 200 nm, and more preferably between 15 nm and 25 nm.

Furthermore, the pores, or holes, can be spaced from each other by a distance of between 100 nm and 1500 nm, for example between 500 nm and 1000 nm.

Thus, the voltage that can be used with a thin layer of nanoporous gold can be between 3V and 5V. In particular, the temperature obtained with the skin device 2 can be between 30° C. and 70° C., preferably between 40° C. and 60° C. In addition, the temperature increase and temperature decrease observed are fast and can thus be perfectly controlled by the voltage applied across the thin metal layer 10.

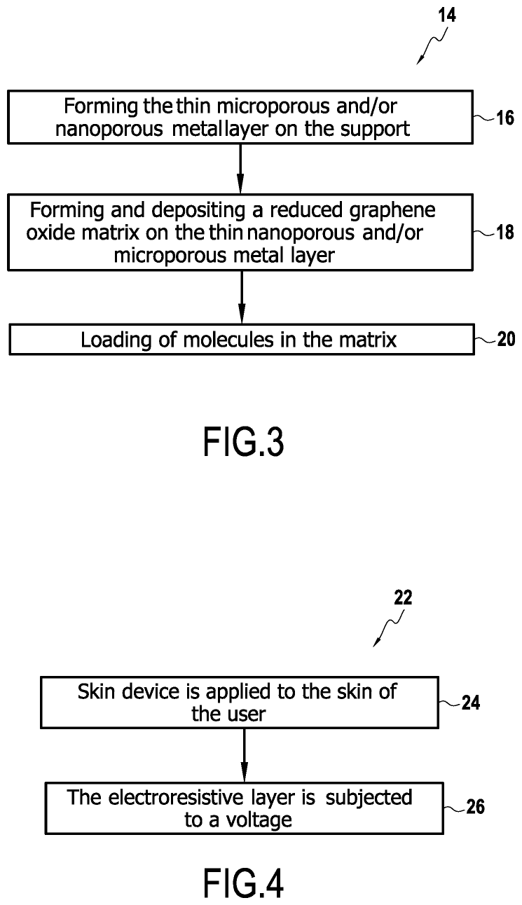

FIG. 3 illustrates a flowchart of a method 14 for manufacturing a cutaneous device 2 as described above. The method 14 thus comprises a first step 16 during which a thin nanoporous metal layer is formed by colloidal lithography.

For example, step 16 may comprise the deposition of a monolayer of polystyrene beads having a diameter of 980 nm on a substrate such as a previously cleaned Kapton® film. The beads are then reduced in size, for example for 11 minutes by etching with oxygen plasma and SF6 at a pressure of 5 mTorr, to a diameter equal, for example, to 630 nm. The sample is then covered with a 2 nm titanium layer and then with a 40 nm gold layer deposited at a constant rate of 2 Å s⁻¹ by vapor deposition. Finally, the polystyrene beads are eliminated by dissolution in chloroform, so as to obtain a thin metal layer consisting of holes having an average diameter of 630 nm and spaced from each other by 980 nm center to center.

The electrical contacts can also be made during the first step 16, for example using masks, so as to obtain an electroresistive layer.

The second step 18 of the method 14 comprises forming and depositing a reduced graphene oxide matrix on the thin metal layer.

Graphene oxide can be synthesized from graphite powder by a modified Hummers method. The graphene oxide thus synthesized is dispersed in water, for example 5 milligrams in 1 ml of water, and exfoliated by ultrasonication for 3 hours. Reduction of graphene oxide to reduced graphene oxide is accomplished by adding hydrazine hydrate (0.50 mL to 32.1 mM) to 5 mL of the aqueous suspension of graphene oxide (0, 5 mg mL−1) and heated at 100° C. for 24 hours. The reduced graphene oxide then gradually precipitates out of the solution: it is isolated by filtration on a polyvinylidene polyfluoride (PVDF) membrane with a pore size of 0.45 μm, washed thoroughly with water and methanol then dried in an oven at 60° C. for 6 hours.

It is also possible to use commercially available reduced graphene oxide.

The reduced graphene oxide thus formed is then deposited on the nanoporous thin metal layer by drop casting (100 μL), three times, followed by drying at room temperature for several hours.

Alternatively, the deposition of the reduced graphene oxide can also be carried out by spin coating, or by electrophoretic deposition or spraying.

The reduced graphene oxide matrix may be loaded with molecules, for example with insulin, before or after its deposition on the nanoporous thin metal layer. For this purpose, the reduced graphene oxide is immersed in a solution containing insulin (500 μg mL⁻¹) and sonicated for 4 hours, and then centrifuged at 13500 rpm for 30 minutes. The insulin concentration stored in the reduced graphene oxide matrix can then be determined by UV/visible spectroscopy, and a loading capacity of 90% is then obtained.

Thus, in a third step 20, the method can provide, if the matrix has not been previously loaded, the loading thereof by the molecules, including insulin, as described above.

FIG. 4 illustrates a flowchart of a method 22 of using a device 1 as described above. The method 22 thus comprises a first step 24 during which the skin device is applied to the skin of the user.

Then, in a second step 26, the electroresistive layer, and more particularly the nanoporous metal thin film, is subjected to a voltage of between 1V and 10V, for example 1V. It is then possible to obtain a temperature close to 60° C. at the level of the nanoporous thin metal layer, in a very short time that can be less than 30 seconds.

The voltage can be applied in response to a command from the individual, or at determined regular intervals.

There is then an insulin release which is particularly effective, of the order of 70% after 5 minutes at a voltage of 1.25V.

Thus, thanks to the use of a thin nanoporous and/or microporous metal layer, it is possible to obtain a flexible cutaneous device that can be heated on demand from a low voltage electrical source. The combination of such an electro-resistive layer with a matrix for storing and releasing molecules, in particular therapeutic molecules, which may have a relatively high molecular weight makes it possible to envisage controlled and easy-to-use transdermal treatments for the users.

### Example

### 1. Experimental Section

1.1. Materials

Graphene oxide (GO) was purchased from Graphenea (Spain).

Kapton® HN Polyimide foils (thickness of 75 and 125 μm) were obtained from DuPont (Circleville, Ohio, USA).

Cafeine, cefepime, 4-fluorouracil, doxorubicin, ampicillin, fluorescence-labeled insulin (FTIC-insulin) were purchased from Sigma-Aldrich.

The human insulin ELISA kit was from Invitrogen (Ref #KAQ1251) and shows an analytical sensitivity of 0.17 μIU/mL and an assay range of 0.1-250 μIU/mL.

1.2. Fabrication of Gold Nanoholes Modified Kapton (K/Au NHs)

Kapton HN polyimide foils (rectangular 20×10 mm² and circular pieces with 20 mm diameter of thicknesses 125 and 75 μm) were cleaned with acetone in an ultrasonic water bath for 30 min, followed with isopropanol for 10 min and then dried under a nitrogen flow. A monolayer of 980 nm polystyrene beads (Microparticles GmbH) was first deposited in the middle of the Kapton surface with an area of 8×10 or 11×11 mm² by self-assembly. To reduce the size of the particles and isolate them, SF₆ and oxygen plasma etching was employed (6 mTorr). The samples were coated with 2 nm Ti followed by 40 nm Au at a constant deposition rate of 1 Å s⁻¹ using physical vapor deposition. The polystyrene beads on top of the Kapton were removed by dissolution in chloroform (overnight).

To facilitate the application of the bias voltage, 2 nm Ti and 40 nm Au contacts were further deposited on both edges of the K/Au NHs patch and two wires 10 cm long were connected (0.34 Ohm cm). The contacts were insulated using epoxy.

The temperature changes were captured by an infrared camera (Thermovision A40) and treated using ThermaCam Researcher Pro 2.9 software

1.3. Preparation of rGO Coated K/Au NHs (K/Au NHs-rGO):

rGO was formed by adding hydrazine hydrate (0.50 mL, 32.1 mM) to 5 mL GO aqueous suspension (0.5 mg mL⁻¹) in a round bottom flask and heated in an oil bath at 100° C. for 24 h. During this time, the reduced GO gradually precipitates out of the solution. The product was isolated by filtration over a polyvinylidene difluoride (PVDF) membrane with a 0.45 μm pore size, washed copiously with water (5×20 mL) and methanol (5×20 mL), and dried in an oven at 60° C. for 6 h. An aqueous suspension of rGO (1 mg mL⁻¹, 5 μL) was drop-casted three times onto K/Au NHs and allowed to dry over night at 70° C. This resulted in a rGO film of about 5 μm.

1.4. Loading of Electrothermal Patch with Drugs

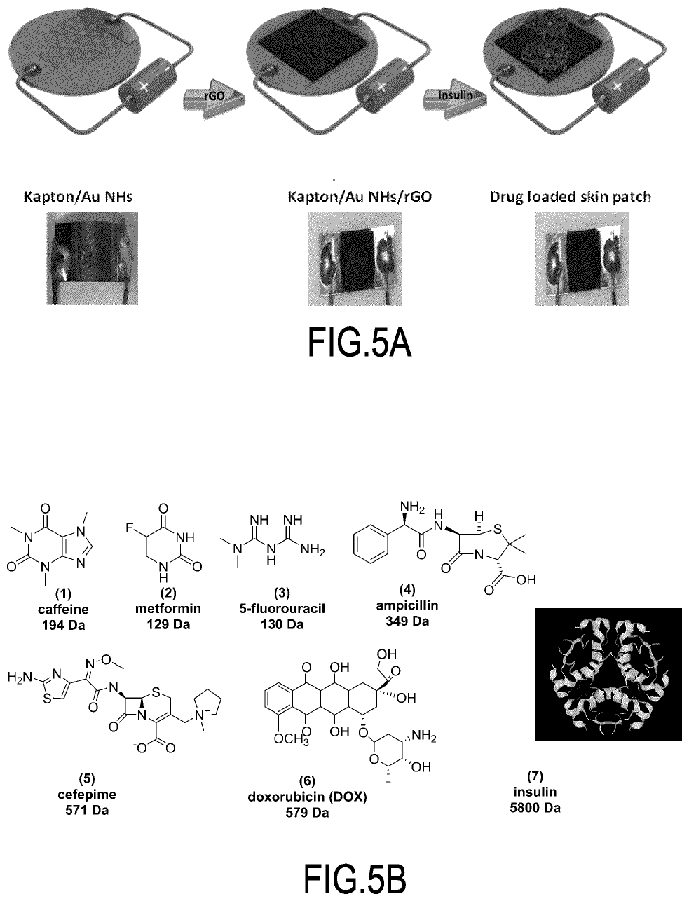

Different drugs (FIG. 5B) (100 μg mL⁻¹ in water) were dropped (30 μL) onto the K/rGO part of the electrothermal patch and left interacting for 2 h at room temperature. Then the patch was rinsed three times with water to wash off any unbound drug.

1.5. Electrothermal Release of Drugs into Solution

Release experiments were performed into 1 mL deionized water. Electrothermal release was done at 1V for 10 min, corresponding to 52±2° C. Thermal images were captured by an Infrared Camera (Thermovision A40) placed 3 cm away from the patch and treated using ThermaCam Researcher Pro 2.9 software. The quantity of drugs released was determined by assessment of drug concentration in the supernatant after activation by HPLC or with Insulin kits. In the case of insulin and DOX, different voltage biases (0.6, 0.8, 0.87, 1 V) were applied to the electrothermal patch for 10 min to investigate the effect of temperature on the skin permeated concentration of insulin and DOX.

1.6. Determination of Loading and Release Capacity of Patch Using HPLC

Different drugs were tested for loading and release. The concentrations of drug loaded and released onto the skin were demined using HPLC. A calibration curve was generated beforehand from a series of drug solutions ranging from 1 to 100 μg/mL. The concentration of the drug in the patch was determined according to equation (1):

[drug]_{rGO}=[drug]ᵢₙᵢₜᵢₐₗ−[drug]ₛᵤₚₑᵣₙₐₜₐₙₜ (1)

with [drug]_{rGO}=concentration of drug on the rGO matrix (μg mL⁻¹)

with [drug]ᵢₙᵢₜᵢₐₗ=concentration of drug in solution (100 μg mL⁻¹)

with [drug]ₛᵤₚₑᵣₙₐₜₐₙₜ=concentration of drug in supernatant (μg mL⁻¹).

HPLC analysis was performed with a Shimadzu LC2010-HT (Shimadzu, Tokyo, Japan) using a C4 QS Uptisphere® column (300 Å, 250×4.6 mm, Interchim, Montluçon, France) heated at 40° C. The mobile phase (A) is trifluoroacetic acid (0.05%) in water and the eluent (B) is trifluoroacetic acid (0.045%) in acetonitrile (flux: 1 mL/min). The flux was: 100% A for 5 min, a linear gradient from 0-80% of B for 15 minutes, then 80% B for 5 min. The detector wavelengths used were 215 nm for ampicillin and 5-fluorouracil, 254 nm for cefepime, 280 nm for cafein and 480 nm for DOX.

1.7. Skin Permeation Experiments

Skin permeation studies were performed using fresh mice skin. For this, mice C57BL/6 were anaesthetized with isoflurane for 10 min, shaved with the use of an electric shaver (Philips Series 7000) and further treated with a depilatory cream (Veet) for 1.5 min. Mice were killed by cervical dislocation and the skin (from the back of mice) was cut into pieces of at least 2 cm in diameter and preserved in DMEM supplemented with gentamicin (0.4%). The thickness of the skin was determined using a digimatic micrometer (Mitutoyo, France) and was determined as 600±5 μm.

A special designed static Franz diffusion cell (ProviSkin, Besancon, France) exhibiting an effective area of 1 cm² and adapted for electrothermal initiated permeability was used for the experiments. The cell comprises two parts. The inside part consists of a circle of 35 mm in outer diameter (7 mm in thickness) and of 19.5 mm inner diameter (6 mm in thickness). The piece is pierced at its center with a hole 1 cm in diameter. Two zones of 5×5 mm2 (6 mm thickness) are cut out at the end of the device to get the connections outside. The Upper part (35 mm in diameter, 9 mm in thickness) comprises a cylinder of 19.5 in diameter and 6 mm in thickness. Holes of 3 mm in diameter are drilled for the electrical contacts of the patch.

After filling the receptor compartment with degassed PBS (1×, pH 7.4), the solution was maintained at 32° C. using a circulating bath (Julabo, France) and stirred with a magnetic stirring bar at around 500 rpm. The mouse skin was carefully clamped between the donor and the receptor compartments (8 mL). Pre-incubation in the receptor compartment medium for 20 min was performed before the drug-loaded electrothermal skin patch was applied to the skin previously wetted with 30 μL of a glycerin solution (50%) to insure contact between the patch and the skin. The system was left for 20 min to be at the right temperature. Electrothermal activation was performed upon continuous application of a power of 1V/0.2 A (corresponding to 50° C.) for 10 min. At determined time intervals (1, 2, 4 and 6 h), 300 μL aliquots of diffused solution were removed from the receptor compartment and analysed. After each sampling, an equal volume of fresh diffusion medium was added to the receptor compartment to maintain a constant volume. All experiments were performed in triplicates.

The release and permeation profiles were determined by plotting the cumulative amount of the drug in the receptor compartment (Qₑₓₚ) (equation 2) against time:

Qₑₓₚ=cₙ×V+Σᵢ₌₁ⁿ⁻¹Vₛ×cᵢ (eq 2)

with Qₑₓₚ=cumulative amount of drug diffused through the skin (μg)
cₙ=amount of drug (μg) determined at the n^{th} sampling interval
V=volume of the acceptor phase (receptor compartment) (mL).

The drug flux (J) was determined according to equation (3):

J=A/S (eq. 3)

with J=flux of drug through the skin (μg cm⁻² h⁻¹)
A=linear slope of the cumulative amount versus time curves in equilibrium conditions (μg h⁻¹)
S=surface of the membrane of the Franz cell (0.64 cm²).

To estimate the amount of drug trapped in the skin, the skin was placed into water/ice mixture for 10 min and sonicated in the presence of ZnO₂ beads (4 mm in diameter), before being centrifuged for 30 min at 13500 rpm using an ultracentrifuge (Mini Scan Fuge ORIGIO). The liquid phase was collected and filtrated through a 0.1 μm Nylon filter (Whatman Puradisc 13 mm) and the amount of drug determined.

1.8. Characterization:

Scanning Electron Microscopy (SEM); Images were obtained using an electron microscope ULTRA 55 (Zeiss) equipped with a thermal field emission emitter and three different detectors (Energy selective Backscattered Detector with filter grid, high efficiency In-lens Secondary Electron Detector and Everhart-Thornley Secondary Electron Detector).

Absorption spectra; Spectra were recorded using a Perkin Elmer Lambda UV-Vis 950 spectrophotometer in a 1-cm quartz cuvette. The wavelength range was 200-1100 nm.

Micro-Raman spectroscopy measurements: Raman spectra were recorded on a Horiba Jobin Yvon LabRam high resolution micro-Raman system combined with a 473-nm (1 mW) laser diode as excitation source. Visible light is focused by a 100× objective. The scattered light is collected by the same objective in backscattering configuration, dispersed by a 1800 mm focal length monochromator and detected by a CCD camera.

1.9. In Vivo Studies

Delivery of insulin from the electrothermal patch was investigated in C57BL/6 mice as model. Before the experiment, the back of the mouse was shaved (Philips Series 7000). The shaved area was further treated with a depilatory cream (Veet) for 1.5 min to eliminate all the remaining hair. The next day the mice fastened for 4 h and the blood glucose level was determined using a glycometer (Roch, France). Before application of the electrothermal patch, the mice are anesthetized with isofluorane for 2 minutes (level 2, 1 l min⁻¹ air) and the blood glucose level was determined again. Then the electrothermal patches loaded with 6 IU (210 μg insulin) were applied to the shaved are and fixed with the help of self-adhesive tape (Sparadrap Micropare, 2133). The patch was activated for 10 min at 1V (corresponding to a surface temperature of 52° C.) and then removed. Blood glucose level values were plotted against time to obtain the blood glucose level-time profiles.

All animal experiments throughout this study were conducted according to the policy of the Federation of European Laboratory Animal Science Association and the European Convention for the protection of vertebrate animals used for experimental and other scientific purposes, with implementation of the principle of the 3Rs (replacement, reduction, refinement).

1.10. Histological Examinations

After C57BL/6 mice were subjected to electrothermal heating, their skin was excised and immerged into formaldehyde (4% in PBS) for 24 h, followed by dehydration (dehydration equipment STP120, Leica) through a graded series of alcohols each for 2 h (70% (2 times) 90% (2 times) 100% (three times). Then, the skin was immersed into xylene (2 times for 2 h) followed by immersion into a paraffin bath of 60° C. (2 times for 2 h). A skin section of 5 μm thickness was cut from the sample (rotary microtom RM2143, Leica) and stained for microscopic examination. Giemsa staining was achieved through the immersion of the skin into: acetone (3 min, 2 times), ethanol (90%, 2 min.), ethanol (70%, 2 min, ethanol (50%, 2 min), ethanol (30%, 2 min), water (2 min.), PBS (pH 6.8, 0.3 M, 5 min), Giemsa ( 1/10 in PBS, 45 min); PBS (pH 6.8, 0.3 M, 2 min), acetic acid (0.15%, 20 sec), water (2 min).

### 2. Results and Discussion

2.1. Flexible Electrothermal Heaters

The electrothermal patch developed in this work is illustrated in FIG. 5. It is based on the formation of gold nanoholes (Au NHs) arrays on Kapton substrate. This substrate has a glass transition between 360° C. and 410° C. and is highly suitable for electrothermal systems. Perforated gold thin films integrated to Kapton (K/Au NHs) are ideal for resistive heating. In contrast to continuous thin gold films, the presence of nanoholes increases the mechanical strength of the metal films.

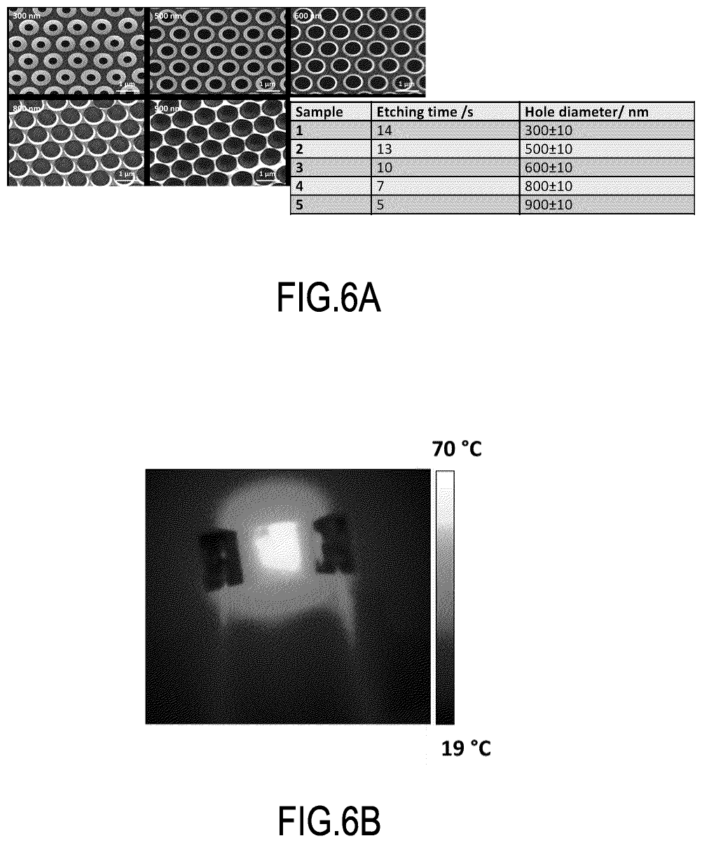

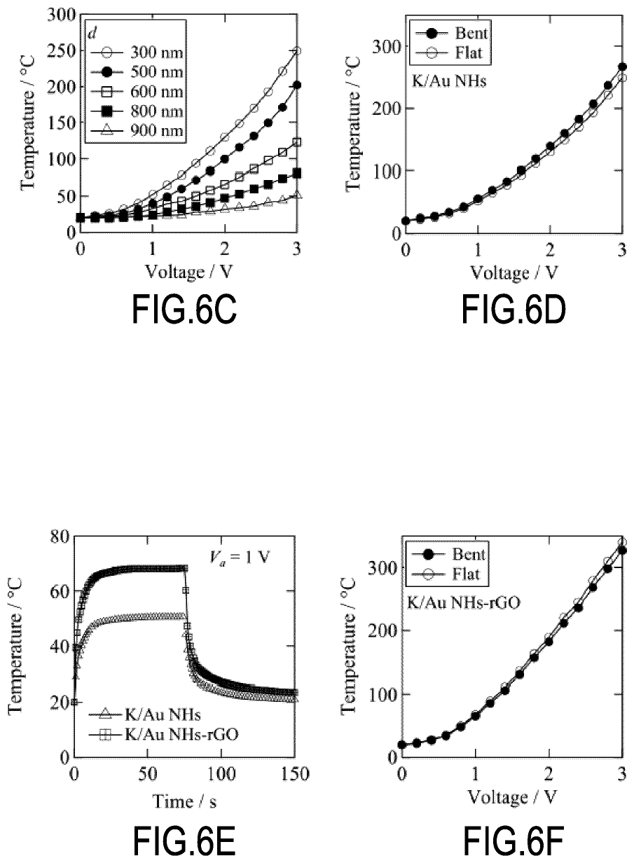

To facilitate the application of different bias voltages, Ti (2 nm)/Au (40 nm) contacting pads were further deposited on the short edges of the patch, to which the electrical power was supplied via connection cables, resulting in homogenous heating profiles. Depending on the etching time used in colloidal lithography, short-ordered holes of varing dimensions were formed (FIG. 6A).

FIG. 6B shows the temperature recording as observed with a thermal camera. A homogenous distribution of the temperature across the surface of the electrothermal patch is measured without the presence of any hot spots, important for an even skin heating. Based on experiments, the best performing electrothermal interface has 300 nm diameter holes (FIG. 6C). An applied voltage of 1V was sufficient enough to raise the temperature to 52° C. steady state in 50 seconds (FIG. 6E) in these devices. Bending the electrothermal patches (60 degrees bending angle) did not affect the performance (FIG. 6D).

For the controlled release of therapeutics, a drug reservoir is required. Reduced graphene oxide was chosen to post coat of the K/Au NHs device due to its excellent drug loading capacity. Deposition of micrometer-thick (5 μm) films of chemically derived rGO onto K/Au NHs results in stable and reproducible electrothermal devices (FIG. 6E, F). Indeed, thickness is the simplest parameter to control the electrical resistance of the coupled metal-rGO system and thus the magnitude of the dc electrical power required to drive the heater to a defined temperature.

3.2. Drug Loading and Release Under Electrothermal Heating

Drug loading was achieved by dropping the drug (100 μg/mL) onto the rGO part of the electrothermal heating surface. Non-covalent interactions such as H-bonding, electrostatic interactions and stacking have been put forward as the main interactions between rGO and the different drugs (FIG. 5B).

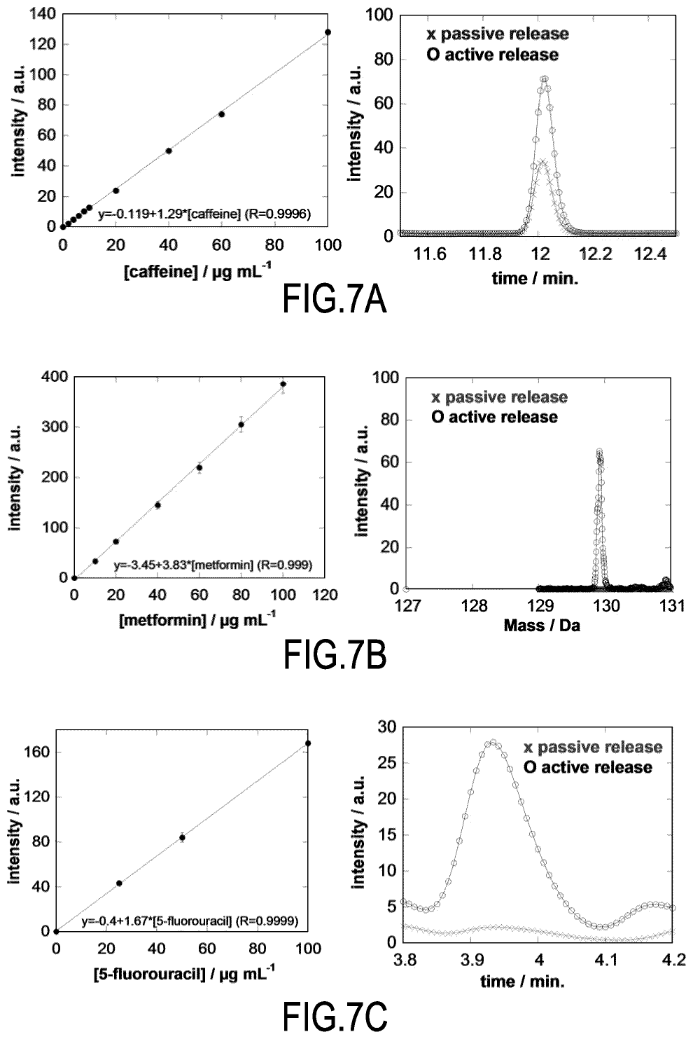

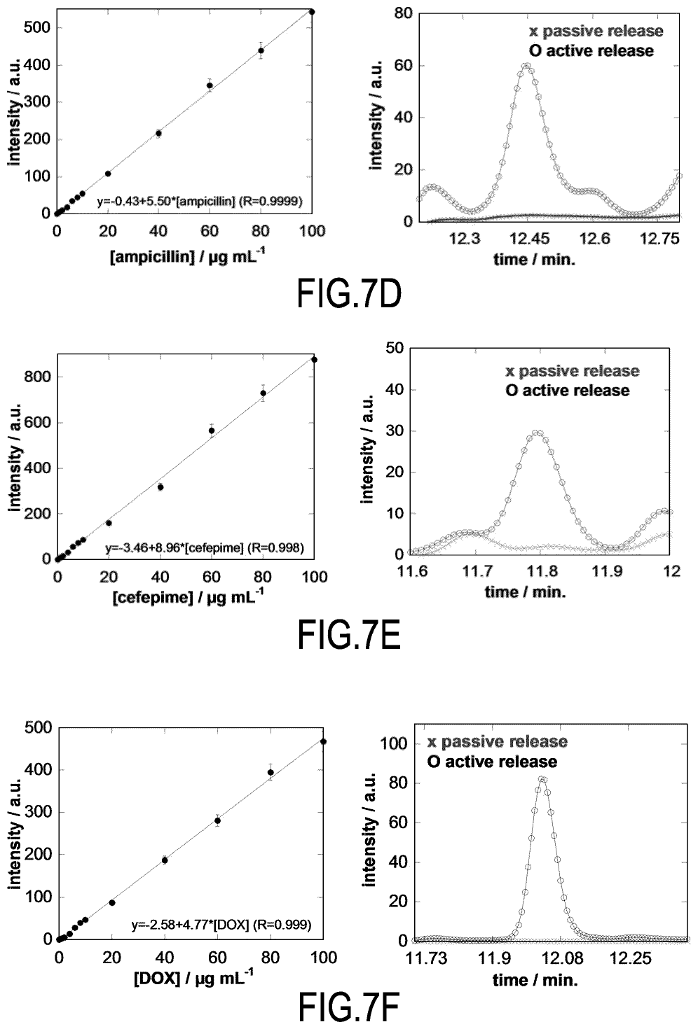

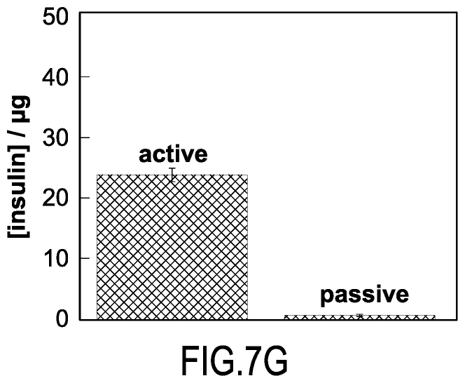

Calibration curves for the different drugs are generated using HPLC signals. The quantities of the drugs delivered through the skin using Franzen diffusion cells using passive and active (electrothermal 1V, 10 min) processes are summarized in FIG. 7. Table 1 summarizes the amount of passed drugs as well as the calculated flux using active and passive modes. In the case of caffeine, while passive transdermal delivery occurs as expected, active delivery reaches caffeine levels which are adequate to elicit ergogenic effects, known to need caffeine concentrations of 5-6 μg/mL. Also passive release is comparable to fluxes reported in the literature. In the case of 5-fluorouracil, no passive transdermal diffusion though the entire skin was observed. However, activation results in a flux of 2 μg cm⁻² h⁻¹. This is higher than the flux of 5-fluoruarcil reported by Xie et al. (1.64 μg cm⁻² h⁻¹) determined from 5-fluoruarcil passed through and remained inside the exercised skin. The amount of ampicillin permeated is above the minimum inhibitory concentration (MIC) of ampicillin for Gram-negative E. coli K12, determined as 10±0.5 μg mL⁻¹ by us lately. The same is true for cefepime, which has a MIC for Gram-negative E. coli K12 of 10±0.5 μg mL⁻¹. In the case of DOX, the possibility of active transdermal delivery was further validated by the fluorescence images where active delivery shows clearly the stratum corneum passage of DOX. The same was true for insulin where FITC-labelled insulin was used for microscopic visualization.

TABLE 1
Amount of drug permeated per square centimeter passively and
through electrothermal activation (1 V, 10 min), a stock
solution of 100 μg mL⁻¹ was used for loading.

| | MW | Passive | Flux passive | Active | Flux active |
|---|---|---|---|---|---|
| drug | [Da] | [μg mL⁻¹] | [μg cm⁻² h⁻¹] | [μg mL⁻¹] | [μg cm⁻² h⁻¹] |
| caffeine | 194 | 28 | 7.18 | 58 | 15.09 |
| metformin | 129 | 0 | 0 | 18 | 4.68 |
| 5-fluorouracil | 130 | 0 | 0 | 23.1 | 6.02 |
| ampicillin, | 349 | 0 | 0 | 17.2 | 4.37 |
| cefepime | 571 | 0 | 0 | 18.3 | 4.67 |
| doxorubicin | 579 | 0 | 0 | 62 | 6.61 |
| insulin | 5800 | 0 | 0 | 24 | 2.60 |

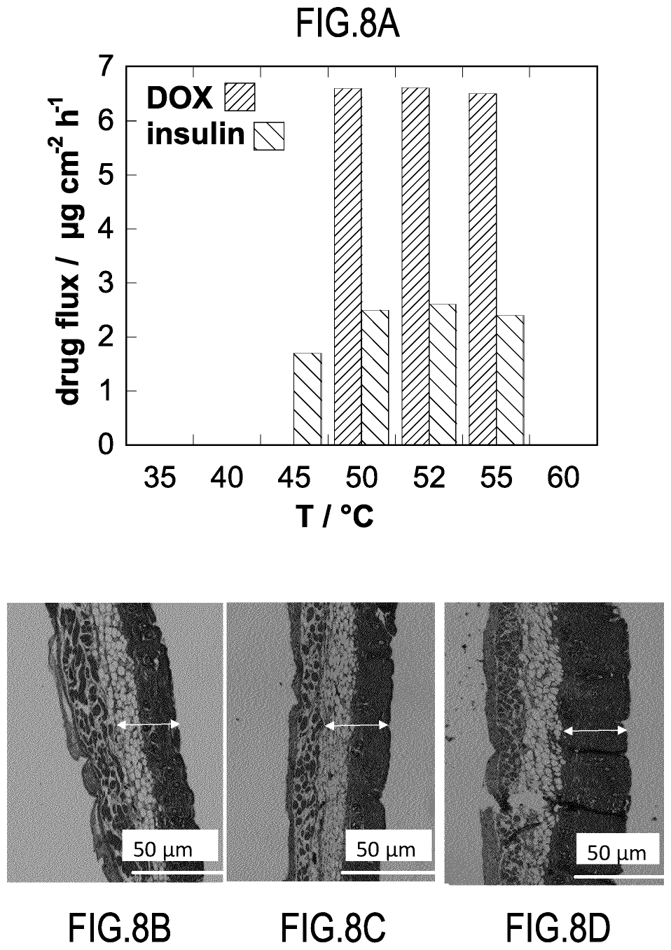

To investigate the influence of temperature (FIG. 8A) the amount of drug (a stock solution of 100 μg mL⁻¹ was used for loading) permeated per square centimeter under electrothermal activation (1V, 10 min) at different time intervals was determined for doxorubicin (DOX) and insulin. In the case of DOX no detective DOX passed at temperature below 50° C., for insulin an increased amount could be detected however. (FIGS. 8B, 8C, 8D)

To further ensure the influence of temperature on the skin structure and eventual inflammation, mouse skin was heated for 10 min at 50, 55 and 60° C. Hematoxylin eosin staining shows intact skin up to 55° C., while at 60° C., thickening of the epidermis and destruction of the sweat gland are observed.

2.6. In Vivo Studies

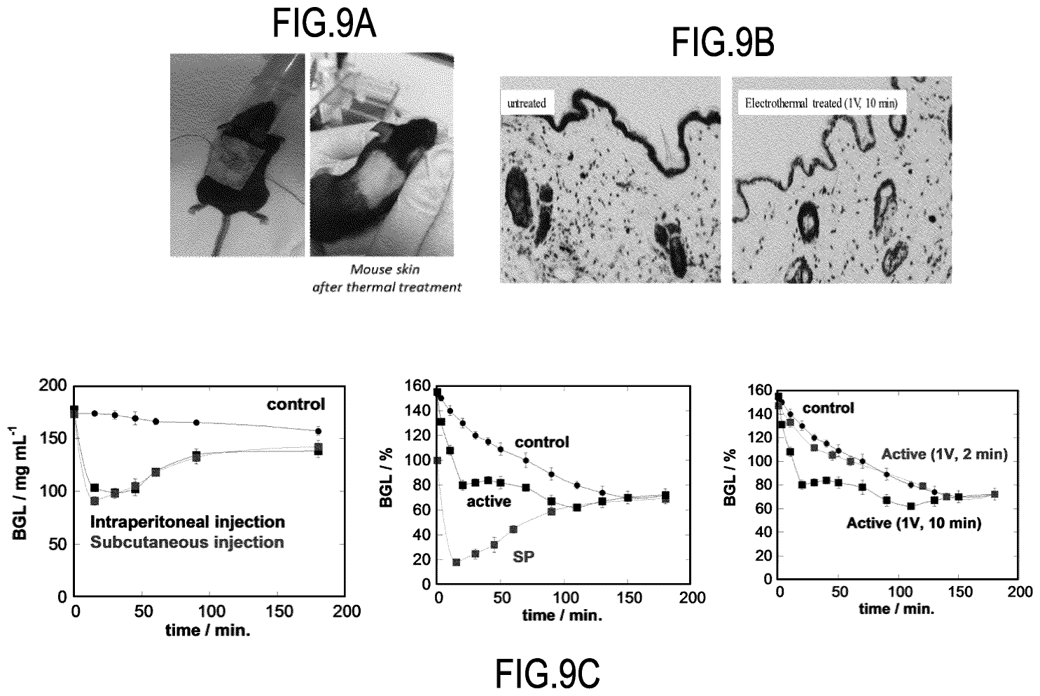

In order to demonstrate the utility of the electrothermal patch to reach therapeutic effects, a range of in vivo experiments was carried out. We used C57BL/6 mice as model in this study and the technical details can be found in the experimental part. Important is to see (FIG. 9A) that after thermal treatment the mouse skin is intact as seen from the photograph of the mouse back and more clearly in the absence of inflamed cells in the histological image. The change in blood glucose level (BGL) among three groups, as shown in FIG. 9B (control, IP injection of insulin and subcutaneous injection of insulin), indicates a therapeutic effect after about 15 min independent on the injection method used. We then applied an electrothermal patch when loaded with 6 IU (210 μg insulin) and when empty (control) and measured the relative BGL level with time (FIG. 9C). A decrease in BGL amount was observed using an insulin loaded and activated patch in the same time scale as for SP injection. Stimulation time has an important effect.

1. A cutaneous device for storing and releasing molecules, in particular therapeutic molecules, comprising:
a support,
a matrix configured to store and release the molecules as a function of the temperature, and
an electroresistive layer disposed between the support and the matrix intended to be subjected to a voltage
wherein the electroresistive layer comprises at least one thin microporous and/or nanoporous metal layer.

2. The cutaneous device according to claim 1, wherein the electroresistive layer is configured to dissipate, as heat, electrical power densities below 200 mW per centimeter square.

3. The cutaneous device according to claim 1, wherein the thin metal layer comprises a material selected from: gold, silver, copper, nickel, platinum, titanium dioxide, and carbon.

4. The cutaneous device according to claim 1, configured to achieve a temperature between 30° C. and 70° C., when the electro-resistive layer is subjected to voltage between 1V and 10V.

5. The cutaneous device according to claim 1, wherein the micrometer and/or nanometer pores have a diameter between 1 nm and 1 μm.

6. The cutaneous device according to claim 1, also comprising the molecules stored and released by the device.

7. The cutaneous device according to claim 1, wherein the molecules are therapeutic molecules having a molecular weight of between 500 Da and 10 kDa.

8. The cutaneous device according to claim 1, wherein the matrix comprises at least reduced graphene oxide or one or more reduced graphene oxide derivatives.

9. The cutaneous device according to claim 1, wherein the support is made of polymer.

10. A device for the transcutaneous release of molecules, in particular therapeutic molecules, comprising a cutaneous device according to claim 1, and means for supplying the electro-resistive layer configured to submit the electro-resistive layer at a voltage of between 1V and 10V.

11. The device according to claim 10, wherein means for supplying the electro-resistive layer are configured to apply a voltage and a current to the electroresistive layer so as to get a dissipation as heat, by the electroresistive layer, of electrical power densities below 200 mW per centimeter square.

12. A method for manufacturing a cutaneous device according to claim 1, comprising: forming the metal thin film by colloidal lithography, on the support and then depositing the matrix configured to store and release molecules, including therapeutic, depending on the temperature, on the thin metal layer.

13. A method of manufacture according to claim 12, wherein, after the deposition of the matrix on the metal thin layer, the molecules are stored in the matrix or in which, before the deposition of the matrix on the thin metallic layer, the molecules are stored in the matrix.

14. A method for transcutaneous delivery of molecules from a cutaneous device according to claim 1, wherein the cutaneous device is applied to the skin of a person, and then subject the electro-resistive layer to a voltage of between 1V and 10V, and to a current so as to get a dissipation, as heat, of electrical power densities below 200 mW per centimeter square.

15. The cutaneous device according to claim 1, wherein the microporous and/or nanoporous thin metallic layer comprises a network of micrometric and/or nanometric pores.

16. The cutaneous device according to claim 6, wherein the molecules stored and released by the device are insulin.

17. The cutaneous device according to claim 9, wherein the support is made of polyimide, polyurethane, polymethylmethacrylate, or polyethylene.

18. The method of manufacture according to claim 12, wherein the matrix configured to store and release molecules, including therapeutic, depending on the temperature, is deposited on the thin metal layer by drop-casting, by spin casting, by electrophoretic deposition or by spraying.

19. The method of manufacture according to claim 12, wherein, before the deposition of the matrix on the thin metallic layer, the molecules are stored in the matrix by implementing the following successive steps:
mixing and stirring of the matrix with the molecules, then
separation by precipitation of the non-stored molecules in the matrix, then
washing of non-stored molecules.
